SEQUENCE LISTING (7 sequences)

SEQ ID NO: 1 - DNA, 1353 bp, Artificial Sequence
  Chimeric antibody
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 2 - DNA, 645 bp, Artificial Sequence
  Chimeric antibody
caaattgttctcacccagtctccagcaatcatgtctgcatctctaggggaacgggtcaccatgacctgcactgccagttcaagtgtaagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 3 - protein, 451 aa, Artificial Sequence
  Chimeric antibody
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 215 aa, Artificial Sequence
  Chimeric antibody
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 696 bp, Artificial Sequence
  Chimeric antibody
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatca

SEQ ID NO: 6 - protein, 232 aa, Artificial Sequence
  Chimeric antibody
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPS

SEQ ID NO: 7 - protein, 5 aa, Artificial Sequence
  Synthetic construct
RCDXC

(19) United States
(12) Patent Application Publication
(10) Pub. No.: US 2011/0110932 A1
(43) Pub. Date: May 12, 2011

(54) COMBINATION TREATMENT FOR OCULAR DISEASES

(75) Inventor: Samir Patel, Princeton, NJ (US)

(73) Assignee: OPHTHOTECH CORP., Princeton, NJ (US)

(21) Appl. No.: 12/768,605

(22) Filed: Apr. 27, 2010

Related U.S. Application Data
(60) Provisional application No. 61/174,971, filed on May 1, 2009.

(51) Int. Cl. A61K 39/395; A61K 38/18; A61K 31/7088

(52) U.S. Cl. 424/133.1; 424/158.1; 424/145.1; 514/8.1; 514/44 R

(57) ABSTRACT

The invention provides compositions and methods for treating ocular disorders, such as angiogenesis-associated disorders, by administering a combination of an inhibitor of VEGF activity and an inhibitor of α₅β₁ integrin activity.

### CROSS REFERENCE TO RELATED APPLICATION

This application claims the benefit of priority under 35 U.S.C. §119(e) to U.S. Provisional Application No. 61/174,971 filed on May 1, 2009, the entire content of which is hereby incorporated herein by reference.

### BACKGROUND OF THE INVENTION

1. Field of the Invention

The invention relates generally to treatment of ocular diseases and more particularly, to compositions and methods of treating angiogenesis-associated ocular disorders.

2. Background Information

Angiogenesis is the process whereby new blood vessels are formed. Angiogenesis, also called neovascularization, occurs normally during embryogenesis and development, and occurs in fully developed organisms during wound healing and placental development. In addition, angiogenesis occurs in various pathological conditions including: ocular diseases such as diabetic retinopathy and macular degeneration due to neovascularization; conditions associated with tissue inflammation such as rheumatoid arthritis and inflammatory bowel disease; and cancer, where blood vessel formation in the growing tumor provides oxygen and nutrients to the tumor cells, as well as providing a route via which tumor cells metastasize throughout the body. Since millions of people around the world are afflicted by these diseases, a considerable effort has been made to understand the mechanisms involved in angiogenesis in order to develop methods for detecting and inhibiting such undesirable angiogenesis.

Angiogenesis occurs in response to stimulation by one or more known growth factors, and also may involve other as yet unidentified factors. Endothelial cells, which are the cells that line mature blood vessels, normally do not proliferate. However, in response to an appropriate stimulus, the endothelial cells become activated and begin to proliferate and migrate into unvascularized tissue to form new blood vessels. In some cases, precursor cells are activated to differentiate into endothelial cells, which form new blood vessels.

Blood vessels are surrounded by an extracellular matrix. In addition to stimulation by growth factors, angiogenesis depends on interaction of the endothelial cells with the extracellular matrix, as well as with each other. The activation of endothelial cells by growth factors and the migration into and interaction with the extracellular matrix and with each other is dependent on cell surface receptors expressed by the endothelial cells. These cell surface receptors, which include growth factor receptors and integrins, interact specifically with particular molecules.

In pathological conditions such as age-related macular degeneration and diabetic retinopathy, decreased availability of oxygen to the retina may result in a hypoxic condition that stimulates the secretion of angiogenic growth factors such as vascular endothelial growth factors (VEGF). This secretion induces abnormal migration and proliferation of endothelial cells into tissues of the eye. This may result in vascularization of ocular tissues and can induce corneal scarring, retinal detachment and fluid accumulation in the choroid, each of which can adversely affect vision and lead to blindness. Thus, a need exists for improved compositions and methods for the treatment of angiogenesis-associated ocular diseases.

### SUMMARY OF THE INVENTION

The present invention is based on the finding that inhibiting both VEGF activity and α₅β₁ integrin activity ameliorates the symptoms associated with angiogenesis-associated ocular disorders. Accordingly, the present invention provides a method of treating an ocular disease. The method includes administering to a subject in need thereof a therapeutically effective amount of an inhibitor of VEGF activity in combination with a therapeutically effective amount of an inhibitor of α₅β₁ integrin activity. In one embodiment, the ocular disease is an angiogenesis-associated ocular disease, such as macular degeneration (including atrophic/dry and exudative/wet macular degeneration), diabetic retinopathy, and choroidal neovascularization.

Both the inhibitor of VEGF activity and the inhibitor of α₅β₁ integrin activity may independently be an antibody, peptide, peptidomimetic, small molecule, chemical or nucleic acid. Exemplary inhibitors of VEGF activity include, but are not limited to, bevacizumab, ranibizumab, pegaptanib sodium, aflibercept, bevasiranib, rapamycin, AGN-745 (an siRNA treatment designed to target vascular endothelial growth factor 1 (VEGF-1); Allergan, Irvine, Calif.), vitalanib, pazopanib, NT-502 (an encapsulated human retinal cells genetically modified to deliver a vascular endothelial growth factor (VEGF) functional antagonist; Neurotech, Lincoln, R.I.), NT-503 (encapsulated human cells genetically modified to deliver a vascular endothelial growth factor (VEGF) structural antagonist; Neurotech, Lincoln, R.I.), and PLG101 (pleiotropic factor acting as an anti-angiogenic factor; PhiloGene, Inc., Summit, N.J.). Exemplary inhibitors of α₅β₁ integrin activity include, but are limited to, volociximab, F200 (a functional fragment of volociximab), 3-(2-{1-alkyl-5-[(pyridine-2-ylamino)-methyl]-pyrrolidin-3-yloxy}-acetylamino)-2-(alkyl-amino)-propionic acid, (S)-2-[(2,4,6-trimethylphenyl)sulfonyl]amino-3-[7-benzyloxycarbonyl-8-(2-pyridinylaminomethyl)-1-oxa-2,7-diazaspiro-(4,4)-non-2-en-3-yl]carbonylamino propionic acid, EMD478761, and the peptide Arg-Cys-Asp-Thioproline-Cys (SEQ ID NO: 7) (RC*D(ThioP)C*, asterisks denote cyclizing by a disulfide bond through the cysteine residues).

The inhibitors of the invention and/or compositions containing the inhibitors of the invention may be administered simultaneously or sequentially via intravitreal or intravenous injection. In one embodiment, the inhibitors are independently administered daily or monthly at doses of about 0.1 mg to about 2.5 mg. In another embodiment, about 0.5 mg of the inhibitor VEGF activity is administered per month. In yet another embodiment, about 0.5 mg, 1.25 mg, or 2.5 mg of the inhibitor of α₅β₁ integrin activity is administered per month. Treatment duration may range from weeks to months to years, including up to three months and/or six months to a year.

The present invention also provides compositions containing the inhibitors of the invention. As such, in one embodiment, the compositions include an inhibitor of VEGF activity and an inhibitor of α₅β₁ integrin activity in a pharmaceutically acceptable carrier. In another embodiment, the compositions include a therapeutically effective amount of ranibizumab and a therapeutically effective amount of volociximab or F200 (a functional fragment of volociximab). In another embodiment, the compositions include a therapeutically effective amount of bevacizumab and a therapeutically effective amount of volociximab or F200 (a functional fragment of volociximab).

Therapeutically effective amounts of the inhibitors in the compositions of the invention include about 0.1 mg to about 6.0 mg of the inhibitor of VEGF activity (e.g., ranibizumab or bevacizumab) and about 0.1 mg to about 2.5 mg of the inhibitor of α₅β₁ integrin activity (e.g., volociximab or F200). In one embodiment, the composition includes therapeutically effective amounts of about 1.0 mg ranibizumab or bevacizumab and about 1.0 mg or about 2.5 mg volociximab or F200.

Therapeutically effective amounts of the inhibitors of the invention range from about 0.1 mg to about 6.0 mg. Thus, in another embodiment, the dosages of the invention include about 0.5 mg of ranibizumab and about 0.5 mg, 1.25 mg, or 2.5 mg of volociximab or F200 (a functional fragment of volociximab). In another embodiment, the dosages of the invention include about 0.5 mg of bevacizumab and about 0.5 mg, 1.25 mg, or 2.5 mg of volociximab or F200.

Concentrations of the inhibitors in the formulations of the invention include about 1 mg/mL to about 60 mg/mL of the inhibitor of VEGF activity (e.g., ranibizumab or bevacizumab) and about 1 mg/mL to about 25 mg/mL of the inhibitor of α₅β₁ integrin activity (e.g., volociximab or F200). In one embodiment, the formulation includes about 10 mg/mL ranibizumab or bevacizumab and about 10 mg/mL or about 25 mg/mL volociximab or F200.

The present invention also provides uses of the inhibitors of the invention and/or compositions of the invention for treating ocular diseases. In one embodiment, the ocular disease is an angiogenesis-associated ocular disease, such as macular degeneration (including atrophic/dry and exudative/wet macular degeneration), diabetic retinopathy, and choroidal neovascularization. The present invention also provides uses of the inhibitors of the invention and/or compositions of the invention in the manufacture of a medicament for treating ocular diseases. In one embodiment, the ocular disease is an angiogenesis-associated ocular disease, such as macular degeneration (including atrophic/dry and exudative/wet macular degeneration), diabetic retinopathy, and choroidal neovascularization.

### BRIEF DESCRIPTION OF THE DRAWINGS

FIGS. 1A and 1B are baseline fluorescein angiography images of the eye of a subject who will undergo combination therapy.

FIGS. 2A-2C are fluorescein angiography images of the eye and fundus of the subject from FIG. 1 at week 5 of combination therapy showing an improvement of +25 letters.

FIGS. 3A-3E are baseline fluorescein angiography images of the eye and fundus of a subject who will undergo combination therapy.

FIGS. 4A-4D are fluorescein angiography images of the eye and fundus of the subject from FIG. 3 at week 9 of combination therapy showing an improvement of +17 letters.

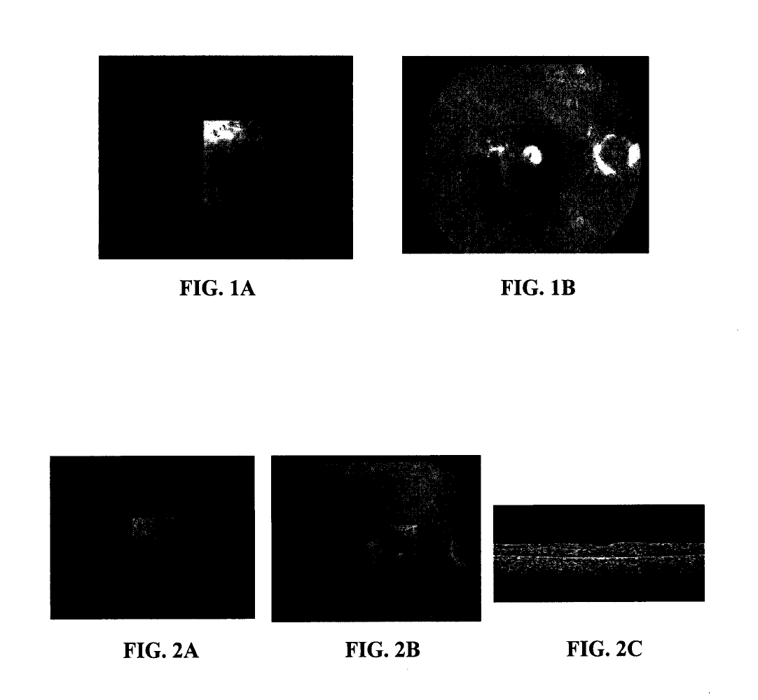

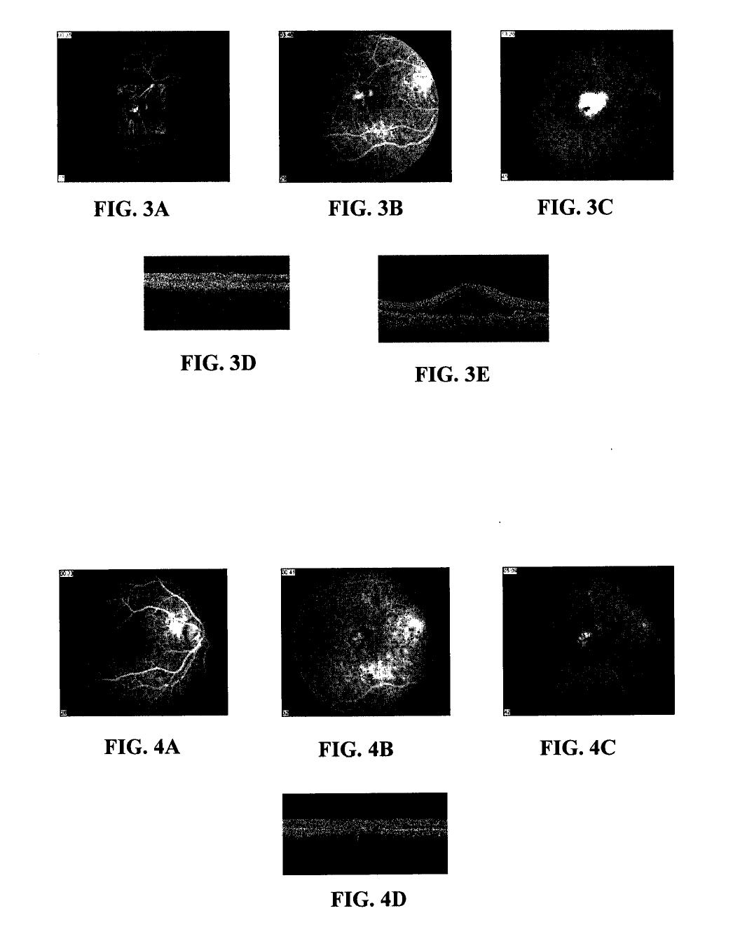

### DETAILED DESCRIPTION OF THE INVENTION

The present invention is based on methods and compositions for treating ocular diseases. In particular, the present invention is based in part on the discovery that co-inhibition of α₅β₁ integrin activity or expression and VEGF activity or expression ameliorates the symptoms associated with angiogenesis-associated ocular diseases such as macular degeneration.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Choroidal neovascularization can lead to hemorrhage and fibrosis, with resulting visual loss in a number of conditions of the eye, including, for example, age-related macular degeneration, ocular histoplasmosis syndrome, pathologic myopia, diabetic retinopathy, angioid streaks, idiopathic disorders, choroiditis, choroidal rupture, overlying choroid nevi, and certain inflammatory diseases. Such disorders will be herein referred to as “ocular diseases”. One of the disorders, namely, age-related macular degeneration (AMD), is the leading cause of severe vision loss in people aged 65 and above.

Deposits under the retina called drusen are a common feature of macular degeneration. Drusen alone usually do not cause vision loss, but when they increase in size or number, this generally indicates an increased risk of developing advanced AMD. As such, macular degeneration can generally be understood to include deterioration or breakdown of the macula. The macula is a small area in the retina at the back of the eye that provides the ability to see fine details clearly. When the macula does not function correctly, central vision can be affected by blurriness, dark areas or distortion. As used herein, the terms “Dry” or “Dry Macular Degeneration” or “Atrophic Macular Degeneration” refer to macular degeneration caused by aging and thinning of the tissues of the macula, which typically results in gradual visual loss. As used herein, the terms “Wet” or “Wet Macular Degeneration” or “Exudative Macular Degeneration” refer to macular degeneration caused by abnormal blood vessels form underneath the retina at the back of the eye. These new blood vessels leak fluid or blood and blur central vision, which typically results in rapid and severe vision loss.

Accordingly, in one aspect, the methods of the invention can be used as part of a treatment regimen for angiogenesis-associated ocular diseases such as macular degeneration. As used herein, an “angiogenesis-associated ocular disease” is any disease or disorder of the eye resulting from undesirable tissue angiogenesis.

Integrins are transmembrane receptors which bind to extracellular matrix proteins and enable not only cell adhesion and cytoskeleton organization, but also transduction of critical signals which promote cell survival, proliferation, differentiation, or migration programs (Mettouchi, et al. European Journal of Cell Biology 85, 243-247, 2006). The integrin family is composed of heterodimers, consisting of one alpha and one beta chain which form non-covalent dimers. Different α and β subunits have been described assembling into more than 24 known endothelial cell adhesion receptors that interact with different and specific ligands from the extracellular matrix (Brooks, et al. Science 264, 569-571, 1994). Of these, Alpha₅Beta₁ (α₅β₁) integrin plays a key role in pathological angiogenesis (Kim, et al. Am. J. Pathol. 156, 1345-1362, 2000). The binding of α₅β₁ integrin to extracellular matrix, specifically fibronectin, leads to intracellular signal transduction controlling critical events involved in angiogenesis (Mettouchi, et al. Mol. Cell 8, 115-127, 2001; Byzova, et al. Mol. Cell 6, 851-860, 2000; and Bayless, et al. Am. J. Pathol. 156, 1673-1683, 2000). These α₅β₁ integrin-mediated activities are downstream to VEGF and other activators of angiogenesis (Kim, et al. Am. J. Pathol. 156, 1345-1362, 2000), making α₅β₁ integrin a target for the treatment of neovascular diseases like wet AMD.

Thus, the invention provides compositions and methods for ameliorating/treating angiogenesis-associated ocular diseases, such as macular degeneration, in a subject. In one embodiment, the method for treating macular degeneration provided herein includes administering to a subject in need thereof an inhibitor of VEGF activity or expression in combination with an inhibitor of α₅β₁ integrin activity or expression.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of the term “subject.”

As used herein, an “inhibitor of VEGF activity or expression” refers to any agent that is capable of inhibiting one or more of the biological activities of VEGF, for example, its angiogenic activity. Antagonists of VEGF act by interfering with formation of VEGF, the binding of VEGF to a cellular receptor, by incapacitating or killing cells which have been activated by VEGF, or by interfering with vascular endothelial cell activation after VEGF binding to a cellular receptor. All such points of intervention by a VEGF antagonist shall be considered equivalent for purposes of this invention. VEGF antagonists useful in the methods of the invention can be any type of molecule, for example, a polynucleotide, a peptide, a peptidomimetic, peptoids such as vinylogous peptoids, nucleic acids, carbohydrates, antibodies, chemicals, a small organic molecule, or any other molecules which reduce, decrease, or otherwise inhibit the normal biological response.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another include, e.g.: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, the terms “reduce” and “inhibit” are used together because it is recognized that, in some cases, a decrease, for example, in VEGF activity can be reduced below the level of detection of a particular assay. As such, it may not always be clear whether the activity is “reduced” below a level of detection of an assay, or is completely “inhibited”. Nevertheless, it will be determinable, following a treatment according to the present methods, that the level of VEGF activity and/or the level of α₅β₁ integrin activity and/or the level of angiogenesis in the particular region or cells being tested is at least reduced from the level before treatment.

The term “VEGF receptor” or “VEGFr” as used herein refers to a cellular receptor for VEGF, ordinarily a cell-surface receptor found on vascular endothelial cells, as well as variants thereof which retain the ability to bind hVEGF. One example of a VEGF receptor is the fms-like tyrosine kinase (fit), a transmembrane receptor in the tyrosine kinase family. DeVries et al., Science 255:989 (1992); Shibuya et al., Oncogene 5:519 (1990). The fit receptor comprises an extracellular domain, a transmembrane domain, and an intracellular domain with tyrosine kinase activity. The extracellular domain is involved in the binding of VEGF, whereas the intracellular domain is involved in signal transduction. Another example of a VEGF receptor is the flk-1 receptor (also referred to as KDR). Matthews et al., Proc. Nat. Acad. Sci. 88:9026 (1991); Terman et al., Oncogene 6:1677 (1991); Terman et al., Biochem. Biophys. Res. Commun. 187:1579 (1992).

In one embodiment, the VEGF anatagonist is an anti-VEGF antibody or functional fragment thereof. Exemplary anti-VEGF antibodies include, but are not limited to, bevacizumab and ranibizumab. Other anti-VEGF antibodies that find use in the compositions and methods of the invention are described in U.S. Pat. Nos. 7,507,405, 7,423,125, 7,422,741, 7,410,639, 7,402,312, 7,375,193, 7,371,377, 7,365,166, 7,297,334, 7,264,801, 7,214,776, 7,208,582, and 7,169,901, the entire content of each of which is incorporated herein by reference.

Additional exemplary VEGF antagonists include, but are not limited to, pegaptanib sodium, aflibercept, bevasiranib, rapamycin, AGN-745 (an siRNA treatment designed to target vascular endothelial growth factor 1 (VEGF-1); Allergan, Irvine, Calif.), vitalanib, pazopanib, NT-502 (an encapsulated human retinal cells genetically modified to deliver a vascular endothelial growth factor (VEGF) functional antagonist; Neurotech, Lincoln, R.I.), NT-503 (encapsulated human cells genetically modified to deliver a vascular endothelial growth factor (VEGF) structural antagonist; Neurotech, Lincoln, R.I.), and PLG101 (pleiotropic factor acting as an anti-angiogenic factor; PhiloGene, Inc., Summit, N.J.).

As used herein, the terms “angiogenesis” and “neoangiogenesis” refer to the formation of new blood vessels, typically in response to insult, injury or disease. For the purposes of this application, the term “injury,” and grammatical variations of the same, includes insult, disease, or other event that results in a tissue response which includes angiogenesis. Angiogenesis also occurs in tumor formation and metastasis, and during embryogenesis, growth and development of higher animals.

As used herein, an “inhibitor of α₅β₁ integrin activity or expression” refers to any agent that is capable of inhibiting one or more of the biological activities of α₅β₁ integrin, for example, its angiogenic activity. Antagonists of α₅β₁ integrin act by interfering with formation of α₅β₁ integrin, the binding of α₅β₁ integrin to a cellular receptor, by incapacitating or killing cells which have been activated by α₅β₁ integrin, or by interfering with vascular endothelial cell activation after α₅β₁ integrin binding to a cellular receptor. All such points of intervention by an α₅β₁ integrin antagonist shall be considered equivalent for purposes of this invention. α₅β₁ integrin antagonists useful in the methods of the invention can be any type of molecule, for example, a polynucleotide, a peptide, a peptidomimetic, peptoids such as vinylogous peptoids, nucleic acids, carbohydrates, antibodies, chemicals, a small organic molecule, or any other molecules which reduce, decrease, or otherwise inhibit the normal biological response.

In one embodiment, the α₅β₁ integrin anatagonist is an anti-α₅β₁ integrin antibody or functional fragment thereof. Exemplary anti-α₅β₁ integrin antibodies include, but are not limited to, volociximab. Volociximab, (also known as M200; PDL BioPharma) is a high affinity chimeric monoclonal antibody which inhibits the binding between α₅β₁ integrin and fibronectin (Varner, et al., Circulation, American Heart Association, 98(17):abstract (1998); Bauer, et al., J. Cell Biol., Vol. 122, No. 1, 20-221, 1993)). At least one function fragment of volociximab (referred to as F200; PDL BioPharma) has also been shown to have α₅β₁ integrin antagonistic activity. F200 is a Fab fragment. Because it is a Fab fragment of volociximab, the F200 light chain DNA and amino acid sequences are the same as the M200 light chain. The complete F200 heavy chain DNA and amino acid sequences are described in U.S. Pat. No. 7,285,268, incorporated herein by reference. The complete DNA and amino acid sequences of M200 and F200 are as follows:

Complete M200 Heavy Chain DNA sequence
(SEQ ID NO: 1):
CAGGTGCAGCTGAAGGAGTCAGGACCTGGCCTGGTGGCGCCCTCACAGA
GCCTGTCCATCACATGCACCATCTCAGGGTTCTCATTAACCGACTATGG
TGTTCACTGGGTTCGCCAGCCTCCAGGAAAGGGTCTGGAGTGGCTGGTA
GTGATTTGGAGTGATGGAAGCTCAACCTATAATTCAGCTCTCAAATCCA
GAATGACCATCAGGAAGGACAACTCCAAGAGCCAAGTTTTCTTAATAAT
GAACAGTCTCCAAACTGATGACTCAGCCATGTACTACTGTGCCAGACAT
GGAACTTACTACGGAATGACTACGACGGGGGATGCTTTGGACTACTGGG
GTCAAGGAACCTCAGTCACCGTCTCCTCAGCTTCCACCAAGGGCCCATC
CGTCTTCCCCCTGGCGCCCTGCTCCAGGAGCACCTCCGAGAGCACAGCC
GCCCTGGGCTGCCTGGTCAAGGACTACTTCCCCGAACCGGTGACGGTGT
CGTGGAACTCAGGCGCCCTGACCAGCGGCGTGCACACCTTCCCGGCTGT
CCTACAGTCCTCAGGACTCTACTCCCTCAGCAGCGTGGTGACCGTGCCC
TCCAGCAGCTTGGGCACGAAGACCTACACCTGCAACGTAGATCACAAGC
CCAGCAACACCAAGGTGGACAAGAGAGTTGAGTCCAAATATGGTCCCCC
ATGCCCATCATGCCCAGCACCTGAGTTCCTGGGGGGACCATCAGTCTTC
CTGTTCCCCCCAAAACCCAAGGACACTCTCATGATCTCCCGGACCCCTG
AGGTCACGTGCGTGGTGGTGGACGTGAGCCAGGAAGACCCCGAGGTCCA
GTTCAACTGGTACGTGGATGGCGTGGAGGTGCATAATGCCAAGACAAAG
CCGCGGGAGGAGCAGTTCAACAGCACGTACCGTGTGGTCAGCGTCCTCA
CCGTCCTGCACCAGGACTGGCTGAACGGCAAGGAGTACAAGTGCAAGGT
CTCCAACAAAGGCCTCCCGTCCTCCATCGAGAAAACCATCTCCAAAGCC
AAAGGGCAGCCCCGAGAGCCACAGGTGTACACCCTGCCCCCATCCCAGG
AGGAGATGACCAAGAACCAGGTCAGCCTGACCTGCCTGGTCAAAGGCTT
CTACCCCAGCGACATCGCCGTGGAGTGGGAGAGCAATGGGCAGCCGGAG
AACAACTACAAGACCACGCCTCCCGTGCTGGACTCCGACGGCTCCTTCT
TCCTCTACAGCAGGCTAACCGTGGACAAGAGCAGGTGGCAGGAGGGGAA
TGTCTTCTCATGCTCCGTGATGCATGAGGCTCTGCACAACCACTACACA
CAGAAGAGCCTCTCCCTGTCTCTGGGTAAA
Complete M200 Light Chain DNA sequence
(SEQ ID NO: 2):
CAAATTGTTCTCACCCAGTCTCCAGCAATCATGTCTGCATCTCTAGGGG
AACGGGTCACCATGACCTGCACTGCCAGTTCAAGTGTAAGTTCCAATTA
CTTGCACTGGTACCAGCAGAAGCCAGGATCCGCCCCCAATCTCTGGATT
TATAGCACATCCAACCTGGCTTCTGGAGTCCCAGCTCGTTTCAGTGGCA
GTGGGTCTGGGACCTCTTACTCTCTCACAATCAGCAGCATGGAGGCTGA
AGATGCTGCCACTTATTACTGCCACCAGTATCTTCGTTCCCCACCGACG
TTCGGTGGAGGCACCAAGCTGGAAATCAAACGAACTGTGGCTGCACCAT
CTGTCTTCATCTTCCCGCCATCTGATGAGCAGTTGAAATCTGGAACTGC
CTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTG
TCACAGAGCAGGACAGCAAGGACAGCACCTACAGCCTCAGCAGCACCCT
GACGCTGAGCAAAGCAGACTACGAGAAACACAAAGTCTACGCCTGCGAA
GTCACCCATCAGGGCCTGAGCTCGCCCGTCACAAAGAGCTTCAACAGGG
GAGAGTGT
Complete M200 Heavy Chain Amino Acid sequence
(SEQ ID NO: 3):
QVQLKESGPGLVAPSQSLSITCTISGFSLTDYGVHWVRQPPGKGLEWLV
VIWSDGSSTYNSALKSRMTIRKDNSKSQVFLIMNSLQTDDSAMYYCARH
GTYYGMTTTGDALDYWGQGTSVTVSSASTKGPSVFPLAPCSRSTSESTA
ALGCLVKDYFPEPVTVSWNSGALTSGVHTFPAVLQSSGLYSLSSVVTVP
SSSLGTKTYTCNVDHKPSNTKVDKRVESKYGPPCPSCPAPEFLGGPSVF
LFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYT
QKSLSLSLGK
Complete M200 Light Chain Amino Acid sequence
(SEQ ID NO: 4):
QIVLTQSPAIMSASLGERVTMTCTASSSVSSNYLHWYQQKPGSAPNLWI
YSTSNLASGVPARFSGSGSGTSYSLTISSMEAEDAATYYCHQYLRSPPT
FGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC
Complete F200 Heavy Chain DNA sequence
(SEQ ID NO: 5):
CAGGTGCAGCTGAAGGAGTCAGGACCTGGCCTGGTGGCGCCCTCACAGA
GCCTGTCCATCACATGCACCATCTCAGGGTTCTCATTAACCGACTATGG
TGTTCACTGGGTTCGCCAGCCTCCAGGAAAGGGTCTGGAGTGGCTGGTA
GTGATTTGGAGTGATGGAAGCTCAACCTATAATTCAGCTCTCAAATCCA
GAATGACCATCAGGAAGGACAACTCCAAGAGCCAAGTTTTCTTAATAAT
GAACAGTCTCCAAACTGATGACTCAGCCATGTACTACTGTGCCAGACAT
GGAACTTACTACGGAATGACTACGACGGGGGATGCTTTGGACTACTGGG
GTCAAGGAACCTCAGTCACCGTCTCCTCAGCTTCCACCAAGGGCCCATC
CGTCTTCCCCCTGGCGCCCTGCTCCAGGAGCACCTCCGAGAGCACAGCC
GCCCTGGGCTGCCTGGTCAAGGACTACTTCCCCGAACCGGTGACGGTGT
CGTGGAACTCAGGCGCCCTGACCAGCGGCGTGCACACCTTCCCGGCTGT
CCTACAGTCCTCAGGACTCTACTCCCTCAGCAGCGTGGTGACCGTGCCC
TCCAGCAGCTTGGGCACGAAGACCTACACCTGCAACGTAGATCACAAGC
CCAGCAACACCAAGGTGGACAAGAGAGTTGAGTCCAAATATGGTCCCCC
ATGCCCATCA
Complete F200 Heavy Chain Amino Acid sequence
(SEQ ID NO: 6):
QVQLKESGPGLVAPSQSLSITCTISGFSLTDYGVHWVRQPPGKGLEWLV
VIWSDGSSTYNSALKSRMTIRKDNSKSQVFLIMNSLQTDDSAMYYCARH
GTYYGMTTTGDALDYWGQGTSVTVSSASTKGPSVFPLAPCSRSTSESTA
ALGCLVKDYFPEPVTVSWNSGALTSGVHTFPAVLQSSGLYSLSSVVTVP
SSSLGTKTYTCNVDHKPSNTKVDKRVESKYGPPCPS

Interestingly, volociximab induces apoptosis in actively proliferating, but not in resting endothelial cells, suggesting that this antibody has a low potential to adversely effect resting endothelium (Ramakrishnan, et al. J Experimental Therap Onc, 5, 273-286, 2006). Both volociximab and F200 showed significant anti-angiogenic activity in choroidal neovascularization (CNV) animal models, including rabbits in which CNV was induced by the suprachoroidal implantation of VEGF/bFGF pellets, and laser induced CNV in cynomolgus monkeys (Id.). Other anti-α₅β₁ integrin antibodies that find use in the compositions and methods of the invention are described in U.S. Pat. Nos. 7,521,425, 7,365,168, 7,285,268, 7,276,589, and 7,153,862, the entire content of each of which is incorporated herein by reference.

Additional exemplary α₅β₁ integrin antagonists include, but are not limited to, 3-(2-{1-alkyl-5-[(pyridine-2-ylamino)-methyl]-pyrrolidin-3-yloxy}-acetylamino)-2-(alkyl-amino)-propionic acid (Umeda, et al., Mol. Pharm., Vol. 69, No. 6, 1820-8, 2006), (S)-2-[(2,4,6-trimethylphenyl)sulfonyl]amino-3-[7-benzyloxycarbonyl-8-(2-pyridinylaminomethyl)-1-oxa-2,7-diazaspiro-(4,4)-non-2-en-3-yl]carbonylamino propionic acid (Maglott, et al., Cancer Res., 66(12), 6002-7, 2006), EMD478761 (a non-peptide diastereomer benzoxazinone molecule described in Fu, et al., Invest. Opthal. Vis. Sci., vol. 48, No. 11, 5184-90, 2007), and the peptide Arg-Cys-Asp-Thioproline-Cys (SEQ ID NO: 7) (RC*D(ThioP)C*, asterisks denote cyclizing by a disulfide bond through the cysteine residues) (Nowlin, et al. J. Biol. Chem., vol. 268, no. 27, 20352-9, 1993).

In another embodiment, the VEGF antagonist and/or the α₅β₁ integrin antagonist may independently be a nucleic acid molecule, such as double-stranded RNA (dsRNA), in order to induce RNA interference (RNAi) and silence VEGF and/or α₅β₁ integrin activity. RNAi is a phenomenon in which the introduction of dsRNA into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. As used herein, “silencing” refers to a mechanism by which cells shut down large sections of chromosomal DNA resulting in suppressing the expression of a particular gene. Without being bound by theory, the RNAi machinery appears to have evolved to protect the genome from endogenous transposable elements and from viral infections. Thus, RNAi can be induced by introducing nucleic acid molecules complementary to the target mRNA to be degraded, as described herein.

In another embodiment, the VEGF antagonist and/or the α₅β₁ integrin antagonist may independently be a designer antibody in which the binding site is engineered to recognize two different antigens (e.g., VEGF and α₅β₁ integrin), both with high affinity (Parren, et al., Science, vol. 323, 1567-8, 2009). As used herein, the term “antibody” includes reference to an immunoglobulin molecule immunologically reactive with a particular antigen, and includes both polyclonal and monoclonal antibodies. The term also includes chimeric antibodies (e.g., humanized murine antibodies) and heteroconjugate antibodies (e.g., bispecific antibodies)). The term “antibody” also includes antigen binding forms of antibodies, including fragments with antigen-binding capability (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3rd Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148: 1547, Pack and Pluckthun (1992) Biochemistry 31: 1579, Gruber et al. (1994) J Immunol:5368, Zhu et al. (1997) Protein Sci 6:781, Hu et al. (1996) Cancer Res. 56: 3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” refer to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

As used herein, a “humanized antibody” refers to an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332: 323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567, incorporated herein by reference), wherein substantially less than an intact human variable domain has been substituted by the corresponding sequence from a non-human species.

As used herein, the terms “epitope” or “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least three, and more usually, at least five or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background.

Specific binding to an antibody under such conditions requires an antibody that is selected for its specificity for a particular protein. For example, antibodies raised against a particular protein, polymorphic variants, alleles, orthologs, and conservatively modified variants, or splice variants, or portions thereof, can be selected to obtain only those polyclonal antibodies that are specifically immunoreactive with α₅β₁ integrin and/or VEGF and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

A number of methods have been described to produce recombinant chimeric antibodies. Controlled rearrangement of antibody domains joined through protein disulfide bonds to form chimeric antibodies can be utilized (Konieczny et al., Haematologia, 14(1):95-99, 1981). Recombinant DNA technology can also be used to construct gene fusions between DNA sequences encoding mouse antibody variable light and heavy chain domains and human antibody light and heavy chain constant domains. See e.g., Morrison et al., Proc. Natl. Acad. Sci. USA, 81(21): 6851-6855, 1984; Morrison, Science 229:1202-1207 (1985); Oi et al., BioTechniques 4:214-221 (1986); Gillies et al., J. Immunol. Methods 125:191-202 (1989); U.S. Pat. Nos. 5,807,715; 4,816,567; and 4,816,397, which are incorporated herein by reference in their entireties.

DNA sequences encoding the antigen binding portions or complementarity determining regions (CDR's) of murine monoclonal antibodies can be grafted by molecular means into the DNA sequences encoding the frameworks of human antibody heavy and light chains (Jones et al., Nature, 321(6069):522-525, 1986; Riechmann et al., Nature, 332(6162):323-327, 1988.). The expressed recombinant products are called “reshaped” or humanized antibodies, and comprise the framework of a human antibody light or heavy chain and the antigen recognition portions, CDR's, of a murine monoclonal antibody.

Another method for producing humanized antibodies is described in U.S. Pat. No. 5,639,641, incorporated herein by reference. The method provides, via resurfacing, humanized rodent antibodies that have improved therapeutic efficacy due to the presentation of a human surface in the variable region. In the method: (1) position alignments of a pool of antibody heavy and light chain variable regions is generated to give a set of heavy and light chain variable region framework surface exposed positions, wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of the complementarity determining regions of the rodent antibody; and (5) the humanized rodent antibody having binding specificity is produced.

A similar method for the production of humanized antibodies is described in U.S. Pat. Nos. 5,693,762; 5,693,761; 5,585,089; and 5,530,101, each incorporated herein by reference. These methods involve producing humanized immunoglobulins having one or more complementarity determining regions (CDR's) and possible additional amino acids from a donor immunoglobulin and a framework region from an accepting human immunoglobulin. Each humanized immunoglobulin chain usually comprises, in addition to the CDR's, amino acids from the donor immunoglobulin framework that are capable of interacting with the CDR's to effect binding affinity, such as one or more amino acids that are immediately adjacent to a CDR in the donor immunoglobulin or those within about 3 Å, as predicted by molecular modeling. The heavy and light chains may each be designed by using any one, any combination, or all of the various position criteria described in U.S. Pat. Nos. 5,693,762; 5,693,761; 5,585,089; and 5,530,101, each of which is incorporated herein by reference. When combined into an intact antibody, the humanized immunoglobulins are substantially non-antibodyic in humans and retain substantially the same affinity as the donor immunoglobulin to the original antigen.

An additional method for producing humanized antibodies is described in U.S. Pat. Nos. 5,565,332 and 5,733,743, each incorporated herein by reference. This method combines the concept of humanizing antibodies with the phagemid libraries also described in detail herein. In a general sense, the method utilizes sequences from the antigen binding site of an antibody or population of antibodies directed against an antigen of interest. Thus for a single rodent antibody, sequences comprising part of the antigen binding site of the antibody may be combined with diverse repertoires of sequences of human antibodies that can, in combination, create a complete antigen binding site.

In one embodiment, the method for treating an ocular disease includes administering to the subject in need thereof a therapeutically effect amount of an inhibitor of VEGF activity in combination with a therapeutically effective amount of an inhibitor of α₅β₁ integrin activity. The term “therapeutically effective amount” or “effective amount” means the amount of a compound or pharmaceutical composition that will elicit the. biological or medical response of a tissue, system, animal or human that is being sought by the researcher, veterinarian, medical doctor or other clinician.

The terms “administration” or “administering” are defined to include the act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravitreal, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

In another embodiment, the present invention provides a method of ameliorating or treating macular degeneration in a subject by administering a therapeutically effective amount of ranibizumab or bevacizumab in combination with a therapeutically effective amount of volociximab or F200. As used herein, the term “ameliorating” or “treating” means that the clinical signs and/or the symptoms associated with an ocular disorder (e.g., macular degeneration) are lessened as a result of the actions performed. The signs or symptoms to be monitored will be characteristic of the ocular disorder (e.g., macular degeneration) and will be well known to the skilled clinician, as will the methods for monitoring the signs and conditions. For example, subjects having macular degeneration may experience fuzzy or blurry vision, an empty or dark area in the center of vision, the appearance of straight lines, such as sides of buildings, telephone poles, or sentences on a page, as curved or wavy, and/or a dimming of vision when reading. Also included in the definition of “ameliorating” or “treating” is the lessening of symptoms associated with the ocular disorders in subjects not yet diagnosed as having the specific disorders. As such, the methods may be used as a means for prophylactic therapy for a subject at risk of having a specific ocular disorder.

In one embodiment, the signs and symptoms associated with an ocular disorder (e.g., macular degeneration) may be monitored by assessment via Optical Coherence Tomography (OCT). OCT is a non-invasive, fast, non-contact imaging technique which readily displays intra-retinal, subretinal and sub-RPE fluid. OCT relies upon differential reflections of light to produce 2-dimensional cross-sections of the retina. OCT images are obtained rapidly and have a spatial resolution of approximately 8 mcm. OCT is especially useful for calculating retinal thickness. In another embodiment, the signs and symptoms associated with an ocular disorder (e.g., macular degeneration) may be monitored by assessment via a visual acuity (VA) test. The visual acuity test is used to determine the smallest letters a person can read on a standardized chart or card held 14-20 feet away. This test is done on each eye, one at a time. If necessary, it is then repeated while the subject wears glasses or contacts.

In certain embodiments, the invention inhibitors may further be administered in combination with an antiinflammatory, antimicrobial, antihistamine, chemotherapeutic agent, antiangiogenic agent, immunomodulator, therapeutic antibody or a protein kinase inhibitor, e.g., a tyrosine kinase inhibitor, to a subject in need of such treatment. Other agents that may be administered in combination with invention compounds include protein therapeutic agents such as cytokines, immunomodulatory agents and antibodies. While not wanting to be limiting, antimicrobial agents include antivirals, antibiotics, anti-fungals and anti-parasitics. When other therapeutic agents are employed in combination with the inhibitors of the present invention they may be used for example in amounts as noted in the Physician Desk Reference (PDR) or as otherwise determined by one having ordinary skill in the art.

The inhibitors of the invention can be administered in any way typical of an agent used to treat the particular type of angiogenesis-associated ocular disorder, or under conditions that facilitate contact of the agent with target intraocular cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

Methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the inhibitor is a small organic molecule such as a steroidal alkaloid, it can be administered in a form that releases the active agent at the desired position in the body (e.g., the eye), or by injection into a blood vessel such that the inhibitor circulates to the target cells (e.g., intraocular cells).

The inhibitors of the invention are also suitably administered by sustained-release systems. Suitable examples of sustained-release compositions include, but are not limited to, semi-permeable polymer matrices in the form of shaped articles, e.g., films, or mirocapsules. Sustained-release matrices include polylactides (U.S. Pat No. 3,773,919, EP 58,481 incorporated herein by reference), copolymers of L-glutamic acid and gamma-ethyl-L-glutamate (U. Sidman et al., Biopolymers 22:547-556 (1983)), poly(2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release VEGF-2 compositions also include liposomally entrapped inhibitors of the invention. Liposomes containing the inhibitors of the invention are prepared by methods known in the art: Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal delivery of the inhibitors of the invention.

In another aspect, the invention provides a method of identifying intraocular cells that are amenable to the treatment methods of the invention. The method can be performed, for example, by measuring the level of VEGF and α₅β₁ integrin expression or activity in a sample of cells to be treated, and determining that VEGF and α₅β₁ integrin expression or activity is elevated as compared to the level of VEGF and α₅β₁ integrin expression or activity in corresponding normal cells, which can be a sample of normal cells (i.e., cells not exhibiting angiogenesis). Alternatively, or in addition, the level of angiogenesis exhibited by the cells in comparison with corresponding normal cells indicates that the cells can benefit from treatment. A sample of cells used in the present method can be obtained from tissue samples or bodily fluid from a subject, or tissue obtained by a biopsy procedure (e.g., a needle biopsy) or a surgical procedure. In one embodiment, the method of identifying intraocular cells amenable to treatment can further include contacting the cells with an inhibitor of VEGF activity in combination with an inhibitor of α₅β₁ integrin activity, and detecting a decrease in VEGF expression or activity and a decrease in α₅β₁ integrin expression or activity following contact. Such a method provides a means to confirm that the intraocular cells are amenable to such treatment.

Accordingly, in another aspect, the methods of the invention are useful for providing a means for practicing personalized medicine, wherein treatment is tailored to a subject based on the particular characteristics of the ocular disorder from which the subject is suffering. The method can be practiced, for example, by contacting a sample of cells from the subject with at least one inhibitor of VEGF expression or activity and at least one inhibitor of α₅β₁ integrin expression or activity, wherein a decrease in VEGF and/or α₅β₁ integrin expression or activity in the presence of the inhibitors as compared to the VEGF and α₅β₁ integrin expression or activity in the absence of the inhibitors identifies the inhibitors as useful for treating the disease. The sample of cells examined according to the present method can be obtained from the subject to be treated, or can be cells of an established ocular disease cell line or known ocular disease of the same type as that of the subject. In one aspect, the established cell line can be one of a panel of such cell lines, wherein the panel can include different cell lines of the same type of disease and/or different cell lines of different diseases associated with angiogenesis. Such a panel of cell lines can be useful, for example, to practice the present method when only a small number of cells can be obtained from the subject to be treated, thus providing a surrogate sample of the subject's cells, and also can be useful to include as control samples in practicing the present methods.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., intraocular fluid, serum, and plasma.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined. In another aspect, the corresponding normal cells comprise a sample of cells obtained from an otherwise healthy portion of tissue of a subject having an ocular disorder.

Once disease is established and a treatment protocol is initiated, the methods of the invention may be repeated on a regular basis to evaluate whether the level of VEGF and/or α₅β₁ integrin expression or activity in the subject begins to approximate that which is observed in a normal subject. Alternatively, or in addition to, the methods of the invention may be repeated on a regular basis to evaluate whether symptoms associated with the particular ocular disease from which the subject suffers have been decreased or ameliorated. The results obtained from successive assays may be used to show the efficacy of treatment over a period ranging from several days to months to years. Accordingly, the invention is also directed to methods for monitoring a therapeutic regimen for treating a subject having an ocular disease. A comparison of the levels of VEGF and α₅β₁ integrin expression or activity and/or a comparison of the symptoms associate with the particular ocular disorder prior to and during therapy indicates the efficacy of the therapy. Therefore, one skilled in the art will be able to recognize and adjust the therapeutic approach as needed.

All methods may further include the step of bringing the active ingredient(s) into association with a pharmaceutically acceptable carrier, which constitutes one or more accessory ingredients. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent.

Thus, in another aspect, the invention provides a formulation including an inhibitor of VEGF activity and an inhibitor of α₅β₁ integrin activity in a pharmaceutically acceptable carrier. In one exemplary embodiment, the concentrations of the inhibitors of the invention include about 1 mg/mL to about 60 mg/mL of inhibitor of VEGF activity (e.g., ranibizumab or bevacizumab) and about 1 mg/mL to about 25 mg/mL of an inhibitor of α₅β₁ integrin activity (e.g., volociximab or F200). In another exemplary embodiment, the concentrations of the inhibitors of the invention include about 1 mg/mL to about 50 mg/mL of inhibitor of VEGF activity (e.g., ranibizumab or bevacizumab) and about 1 mg/mL to about 25 mg/mL of an inhibitor of α₅β₁ integrin activity (e.g., volociximab or F200). In another exemplary embodiment, the concentration of the inhibitors of the invention includes about 10 mg/mL ranibizumab or bevacizumab and about 10 mg/mL or about 25 mg/mL volociximab or F200.

The volociximab or F200 may be contained in an aqueous solution that includes about 22 mM to about 27 mM citrate, about 145 mM to about 165 mM sodium chloride, about 0.04% to about 0.06% polysorbate 80, and a pH of about 5.5 to 7.5. In one embodiment, the volociximab solution contains 25 mM citrate, 150 mM sodium chloride, 0.05% polysorbate 80, at pH 6.5. Likewise, the ranibizumab may be contained in an aqueous solution that includes about 10 mM histidine HCl, about 10% a,a-trehalose dehydrate, and about 0.01% polysorbate 20, at pH 5.5. Finally, a 100 mg aqueous solution of bevacizumab may contain 240 mg α,α-trehalose dehydrate, 23.2 mg sodium phosphate (monobasic, monohydrate), 4.8 mg sodium phosphate (dibasic, anhydrous), 1.6 mg polysorbate 20, and water for injection. As such, one of skill in the art will recognize that the pharmaceutically acceptable carrier in the composition of the invention may further include one or more of the above-described excipients.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the inhibitor of VEGF expression or activity and the amount of the inhibitor of α₅β₁ integrin expression or activity to treat ocular disorders in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

Accordingly, in certain embodiments, the methods of the invention include an intervalled or sequential treatment regimen. Without being bound to theory, it may be observed that one of the inhibitors of the invention has a longer half-life, for example, and therefore accumulates in the subject being treated. As such, intervalled administration allows for less frequent administrations of any inhibitor, while the other inhibitor is administered at more frequent intervals. Thus, in one exemplary embodiment, the inhibitor of VEGF activity and the inhibitor of α₅β₁ integrin activity are administered sequentially. In another exemplary embodiment, inhibitor of VEGF activity and the inhibitor of α₅β₁ integrin activity are administered simultaneously. Treatment duration may range from weeks to months to years, including up to three months and/or up to six months to a year.

In another embodiment, each administration (e.g., intravitreal injection) provides a dosage of about 0.1 mg to about 6.0 mg of each inhibitor. In another embodiment, each administration (e.g., intravitreal injection) provides about 0.5 mg, 1.0 mg, 1.5 mg, 2.0 mg, 2.5 mg, 3.0 mg, 3.5 mg, 3.5 mg, 4.0 mg, 4.5 mg, 5.0 mg, 5.5 mg, or 6.0 mg of the inhibitor VEGF activity. In yet another embodiment, each administration (e.g., intravitreal injection) provides about 0.5 mg, 1.25 mg, or 2.5 mg of the inhibitor of α₅β₁ integrin activity.

In another embodiment, the inhibitors of the invention are administered daily or monthly as part of a composition. As such, the therapeutically effective dose of each of the inhibitors in the compositions may independently be about 0.1 mg to about 6.0 mg. In one embodiment, the therapeutically effective dose of each of the inhibitors is about 0.1 mg to about 2.5 mg.

In another aspect, the invention provides kits for performing the methods of the invention that include at least one inhibitor VEGF activity or expression and at least one inhibitor of α₅β₁ integrin expression or activity in a pharmaceutically acceptable carrier. In one embodiment, the invention provides a kit that includes a pharmaceutical composition comprising an inhibitor of VEGF activity or expression and an inhibitor of α₅β₁ integrin activity or expression. The included inhibitors may be antibodies, such as volociximab or F200 and ranibizumab, or may be a dsRNA that hybridizes to a polynucleotide encoding or regulating VEGF or a functional fragment thereof, and a dsRNA that hybridizes to a polynucleotide encoding or regulating α₅β₁ integrin or a functional fragment thereof. In another embodiment, the kit includes instructions for practicing the methods of the invention.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Combination Therapy

This example demonstrates the safety of intravitreal volociximab, an α5β1 integrin antagonist, in combination with ranibizumab, to subjects with wet AMD.

Volociximab, an anti-α5β1 integrin monoclonal antibody, has demonstrated potent anti-angiogenic effects in various in vitro models, including inhibition of human umbilical vein endothelial cell (HUVEC) proliferation and HUVEC tube formation. As such, the invention demonstrates a safety study of volociximab in combination with ranibizumab.

Bulk 25 mg/mL volociximab (also known as M200; PDL BioPharma) is aseptically filled into clinical use vials. A 10 mg/mL solution is prepared by dilution of the 25 mg/mL bulk using the same formulation solution. Stock ranibizumab (Genentech) at 10 mg/mL is aseptically filled into clinical use vials.

This is a dose-escalating, uncontrolled, multiple-dose multicenter study including subfoveal lesions of any subtype secondary to AMD. Subjects are enrolled in a dose escalation scheme to receive three monthly intravitreal administrations of volociximab in treatment-naïve eyes at one of three doses (up to six subjects per group receiving either 0.5, 1.25 or 2.5 mg in ascending order) in combination with monthly ranibizumab at 0.5 mg. Thereafter, approximately 30 subjects (anti-VEGF experienced eyes or treatment-naive eyes) will receive three monthly doses of volociximab (either 1.25 mg or 2.5 mg) given in combination with ranibizumab 0.5 mg.

Ten subjects have received two doses of volociximab in combination with ranibizumab for wet AMD. Baseline visual acuity (VA) and optical coherence tomography (OCT) center point thickness were 54 letters and 366 μm, respectively. Results of visual acuity testing after two doses of combination therapy (week 9) revealed a mean change in VA of +10.8 letters, with 40% of patients gaining ≧3 lines. The mean change in OCT center point thickness was −136 μm. Dose escalation has been completed without evidence of dose-limiting toxicity or drug-related adverse events. Results of the initial ten subjects that received two doses of volociximab in combination with ranibizumab for wet AMD suggest that volociximab has a favorable safety profile.

Although the invention has been described with reference to the above example, it will be understood that modifications and variations are encompassed within the spirit and scope of the invention. Accordingly, the invention is limited only by the following claims.

What is claimed is:

1. A method of treating an ocular disease comprising administering to a subject in need thereof a therapeutically effective amount of an inhibitor of VEGF activity in combination with a therapeutically effective amount of an inhibitor of α₅β₁ integrin activity.

2. The method of claim 1, wherein the ocular disease is an angiogenesis-associated ocular disease.

3. The method of claim 2, wherein the ocular disease is selected from the group consisting of macular degeneration, diabetic retinopathy, and choroidal neovascularization.

4. The method of claim 3, wherein the ocular disease is wet macular degeneration.

5. The method of claim 3, wherein the ocular disease is dry macular degeneration.

6. The method of claim 1, wherein administering comprises intravitreal injection.

7. The method of claim 1, wherein administering comprises intravenous injection.

8. The method of claim 1, wherein the inhibitor of VEGF activity is an antibody or functional fragment thereof.

9. The method of claim 8, wherein the inhibitor of VEGF activity is bevacizumab or ranibizumab.

10. The method of claim 1, wherein the inhibitor of VEGF activity is a peptide, peptidomimetic, small molecule, chemical or nucleic acid.

11. The method of claim 10, wherein the inhibitor of VEGF activity is pegaptanib sodium, aflibercept, bevasiranib, rapamycin, AGN-745, vitalanib, pazopanib, NT-502, NT-503, or PLG101.

12. The method of claim 1, wherein the inhibitor of α₅β₁ integrin activity is an antibody or functional fragment thereof.

13. The method of claim 12, wherein the inhibitor of α₅β₁ integrin activity is volociximab.

14. The method of claim 1, wherein the inhibitor of α₅β₁ integrin activity is a peptide, peptidomimetic, small molecule, chemical or nucleic acid.

15. The method of claim 14, wherein the inhibitor of α₅β₁ integrin activity is 3-(2-{1-alkyl-5-[(pyridine-2-ylamino)-methyl]-pyrrolidin-3-yloxy}-acetylamino)-2-(alkyl-amino)-propionic acid, (S)-2-[(2,4,6-trimethylphenyl)sulfonyl]amino-3-[7-benzyloxycarbonyl-8-(2-pyridinylaminomethyl)-1-oxa-2,7-diazaspiro-(4,4)-non-2-en-3-yl]carbonylamino propionic acid, EMD478761, or RC*D(ThioP)C*.

16. The method of claim 1, wherein the inhibitor of VEGF activity and the inhibitor of α₅β₁ integrin activity are administered simultaneously.

17. The method of claim 1, wherein the inhibitor of VEGF activity and the inhibitor of α₅β₁ integrin activity are administered sequentially.

18. The method of claim 1, wherein the inhibitor of VEGF activity is administered daily.

19. The method of claim 1, wherein the inhibitor of VEGF activity is administered monthly.

20. The method of claim 1, wherein the inhibitor of α₅β₁ integrin activity is administered daily.

21. The method of claim 1, wherein the inhibitor of α₅β₁ integrin activity is administered monthly.

22. The method of claim 1, wherein about 0.1 mg to about 6.0 mg of the inhibitor VEGF activity is administered per month.

23. The method of claim 22, wherein about 0.5 mg of the inhibitor VEGF activity is administered per month.

24. The method of claim 1, wherein about 0.1 mg to about 2.5 mg of the inhibitor of α₅β₁ integrin activity is administered per month.

25. The method of claim 24, wherein about 0.5 mg, 1.25 mg, or 2.5 mg of the inhibitor of α₅β₁ integrin activity is administered per month.

26. The method of claim 1, wherein treatment duration is up to three months.

27. The method of claim 1, wherein treatment duration is six months to a year.

28. The method of claim 1, wherein the subject is a mammal.

29. The method of claim 28, wherein the subject is human.

30. A method of treating macular degeneration comprising administering to a subject in need thereof a therapeutically effective amount of ranibizumab or bevacizumab in combination with a therapeutically effective amount of volociximab.

31. The method of claim 30, wherein the volociximab is administered monthly via intravitreal injection.

32. The method of claim 31, wherein about 0.1 mg to about 2.5 mg of volociximab is administered in each injection.

33. The method of claim 32, wherein about 0.5 mg, 1.25 mg, or 2.5 mg of volociximab is administered in each injection.

34. The method of claim 30, wherein the ranibizumab or bevacizumab is administered monthly via intravitreal injection.

35. The method of claim 34, wherein about 0.1 mg to about 6.0 mg of ranibizumab or bevacizumab is administered in each injection.

36. The method of claim 35, wherein about 0.5 mg of ranibizumab or bevacizumab is administered in each injection.

37. The method of claim 30, wherein the subject is a mammal.

38. The method of claim 37, wherein the subject is human.

39. A composition comprising an inhibitor of VEGF activity and an inhibitor of α₅β₁ integrin activity in a pharmaceutically acceptable carrier.

40. The composition of claim 39, wherein the inhibitor of VEGF activity is bevacizumab, ranibizumab, pegaptanib sodium, aflibercept, bevasiranib, rapamycin, AGN-745, vitalanib, pazopanib, NT-502, NT-503, or PLG101.

41. The composition of claim 39, wherein the inhibitor of α₅β₁ integrin activity is volociximab, 3-(2-{1-alkyl-5-[(pyridine-2-ylamino)-methyl]pyrrolidin-3-yloxy}-acetylamino)-2-(alkyl-amino)-propionic acid, (S)-2-[(2,4,6-trimethylphenyl)sulfonyl]amino-3-[7-benzyloxycarbonyl-8-(2-pyridinylaminomethyl)-1-oxa-2,7-diazaspiro-(4,4)-non-2-en-3-yl]carbonylamino propionic acid, EMD478761, or RC*D(ThioP)C*.

42. A composition comprising a therapeutically effective amount of ranibizumab or bevacizumab and a therapeutically effective amount of volociximab in a pharmaceutically acceptable carrier.

43. The composition of claim 42, wherein the therapeutically effective amount of ranibizumab or bevacizumab is about 0.1 mg to about 6.0 mg.

44. The composition of claim 42, wherein the therapeutically effective amount of volociximab is about 0.1 mg to about 2.5 mg.

45. The composition of claim 44, wherein the therapeutically effective amount of volociximab is about 1.0 mg or about 2.5 mg.

46. The composition of claim 42, wherein the therapeutically effective amount of ranibizumab or bevacizumab is about 0.1 mg to about 6.0 mg.

47. The composition of claim 42, wherein the therapeutically effective amount of ranibizumab or bevacizumab is about 1.0 mg.

48. A composition comprising about 0.1 mg to about 6.0 mg ranibizumab or bevacizumab and about 0.1 mg to about 2.5 mg volociximab in a pharmaceutically acceptable carrier.

49. A composition comprising about 0.5 mg of ranibizumab or bevacizumab and about 0.5 mg, 1.25 mg, or 2.5 mg of volociximab in a pharmaceutically acceptable carrier.

50. A composition comprising a therapeutically effective amount of about 1.0 mg ranibizumab or bevacizumab and a therapeutically effective amount of about 1.0 mg or about 2.5 mg volociximab.
